(19) United States
(12) Patent Application Publication
(10) Pub. No.: US 2025/0037806 A1
(43) Pub. Date: Jan. 30, 2025

(54) METHOD, APPARATUS, DEVICE AND MEDIUM FOR MANAGING MOLECULAR PREDICTION

(71) Applicants: Beijing Bytedance Network Technology Co., Ltd., Beijing (CN); Lemon Inc., Grand Cayman (KY)

(72) Inventors: Xiang GAO, Los Angeles, CA (US); Weihao GAO, Beijing (CN); Wenzhi XIAO, Beijing (CN); Zhirui WANG, Los Angeles, CA (US); Liang XIANG, Beijing (CN); Chong WANG, Los Angeles, CA (US)

(21) Appl. No.: 18/717,362

(22) PCT Filed: Apr. 20, 2023
(86) PCT No.: PCT/CN2023/089548

§ 371 Date: Jun. 6, 2024

(30) Foreign Application Priority Data
May 13, 2022 (CN) 202210524875.6

(51) Int. Cl. G16C 20/70

(52) U.S. Cl. CPC G16C 20/70

(57) ABSTRACT

According to implementations of the present disclosure, a method, apparatus, device and medium for managing molecular prediction is provided. In the method, an upstream model is obtained from a portion of network layers in a pretrained model, the pretrained model describing an association between a molecular structure and molecular energy. A downstream model is determined based on a molecular prediction purpose, and an output layer of the downstream model is determined based on the molecular prediction purpose. A molecular prediction model is generated based on the upstream model and the downstream model, the molecular prediction model describing an association between a molecular structure and a molecular prediction purpose associated with the molecular structure. Since the upstream model may have extensive knowledge related to molecules, the amount of training data required to train the molecular prediction model that is generated based on the upstream model and the downstream model may be reduced.

This application claims priority to Chinese Patent Application No. 202210524875.6, filed with the Chinese Patent Office on May 13, 2022, and entitled “METHOD, APPARATUS, DEVICE AND MEDIUM FOR MANAGING MOLECULAR PREDICTION”, the disclosures of which are incorporated herein by reference in their entities.

### FIELD

Example implementations of the present disclosure generally relate to the computer field, and particularly to, a method, apparatus, device and computer readable storage medium for managing molecular prediction.

### BACKGROUND

With the development of machine learning technology, machine learning technology has been widely used in various technical fields. Molecular research is an important task in fields such as materials science, energy applications, biotechnology, and pharmaceutical research. Machine learning has been widely applied in these fields and may predict features of other molecules based on known molecular features. However, machine learning technology relies on a large amount of training data, and the collection of training datasets requires a lot of experiments and consumes a lot of manpower, material resources, and time. At this point, how to improve the precision of prediction models in the absence of sufficient training data has become a difficult and hot topic in the field of molecular research.

### SUMMARY

According to implementations of the present disclosure, a solution for managing molecular prediction is provided.

In a first aspect of the present disclosure, a method for managing molecular prediction is provided. In the method, an upstream model is obtained from a portion of network layers in a pretrained model, the pretrained model describing an association between a molecular structure and molecular energy. A downstream model is determined based on a molecular prediction purpose, and an output layer of the downstream model is determined based on the molecular prediction purpose. A molecular prediction model is generated based on the upstream model and the downstream model, the molecular prediction model describing an association between a molecular structure and a molecular prediction purpose associated with the molecular structure.

In a second aspect of the present disclosure, an apparatus for managing molecular prediction is provided. The apparatus comprises: an obtaining module configured for obtaining an upstream model from a portion of network layers in a pretrained model, the pretrained model describing an association between a molecular structure and molecular energy: a determining module configured for determining a downstream model based on a molecular prediction purpose, and an output layer of the downstream model being determined based on the molecular prediction purpose; and a generating module configured for generating a molecular prediction model based on the upstream model and the downstream model, the molecular prediction model describing an association between a molecular structure and a molecular prediction purpose associated with the molecular structure.

In a third aspect of the present disclosure, an electronic device is provided. The electronic device comprises: at least one processing unit; and at least one memory, coupled to the at least one processing unit and storing instructions executed by the at least one processing unit, the instructions, when executed by the at least one processing unit, causing the electronic device to perform the method according to the first aspect of the present disclosure.

In a fourth aspect of the present disclosure, a computer-readable storage medium is provided, storing a computer program thereon, the computer program, when executed by a processor, causing the processor to implement the method according to the first aspect of the present disclosure.

It should be understood that what is described in this Summary is not intended to identify key features or essential features of the implementations of the present disclosure, nor is it intended to limit the scope of the present disclosure. Other features disclosed herein will become easily understandable through the following description.

### BRIEF DESCRIPTION OF THE DRAWINGS

The above and other features, advantages, and aspects of respective implementations of the present disclosure will become more apparent from the following detailed description with reference to the accompanying drawings. The same or similar reference numerals represent the same or similar elements throughout the figures, where:

FIG. 1 illustrates a block diagram of an example environment in which the implementations of the present disclosure may be implemented;

FIG. 2 illustrates a block diagram of a process for managing molecular prediction according to some implementations of the present disclosure:

FIG. 3 illustrates a block diagram of the process for generating a molecular prediction model based on a pretrained model according to some implementations of the present disclosure:

FIG. 4 illustrates a block diagram of a process for obtaining a pretrained model according to some implementations of the present disclosure:

FIG. 5 illustrates a block diagram of a loss function for a pretrained model according to some implementations of the present disclosure:

FIG. 6 illustrates a block diagram of a process for obtaining a molecular prediction model according to some implementations of the present disclosure:

FIG. 7 illustrates a block diagram of a loss function for a molecular prediction model according to some implementations of the present disclosure:

FIG. 8 illustrates a flowchart of a method for managing molecular prediction according to some implementations of the present disclosure:

FIG. 9 illustrates a block diagram of an apparatus for managing molecular prediction according to some implementations of the present disclosure; and

FIG. 10 illustrates a block diagram of a device capable of implementing a plurality of implementations of the present disclosure.

### DETAILED DESCRIPTION

The implementations of the present disclosure will be described in more detail with reference to the accompanying drawings, in which some implementations of the present disclosure have been illustrated. However, it should be understood that the present disclosure may be implemented in various manners, and thus should not be construed to be limited to implementations disclosed herein. On the contrary, those implementations are provided for the thorough and complete understanding of the present disclosure. It should be understood that the drawings and implementations of the present disclosure are only used for illustration, rather than limiting the protection scope of the present disclosure.

As used herein, the term “comprise” and its variants are to be read as open terms that mean “include, but is not limited to.” The term “based on” is to be read as “based at least in part on.” The term “one implementation” or “the implementation” is to be read as “at least one implementation.” The term “some implementations” is to be read as “at least some implementations.” Other definitions, explicit and implicit, might be further included below: As used herein, the term “model” may represent associations between respective data. For example, the above association may be obtained based on various technical solutions that are currently known and/or to be developed in future.

It is to be understood that the data involved in this technical solution (including but not limited to the data itself, data acquisition or use) should comply with the requirements of corresponding laws and regulations and relevant provisions.

It is to be understood that, before applying the technical solutions disclosed in respective embodiments of the present disclosure, the user should be informed of the type, scope of use, and use scenario of the personal information involved in the present disclosure in an appropriate manner in accordance with relevant laws and regulations, and user authorization should be obtained.

For example, in response to receiving an active request from the user, prompt information is sent to the user to explicitly inform the user that the requested operation would acquire and use the user's personal information. Therefore, according to the prompt information, the user may decide on his/her own whether to provide the personal information to the software or hardware, such as electronic devices, applications, servers, or storage media that perform operations of the technical solutions of the present disclosure.

As an optional but non-limiting implementation, in response to receiving an active request from the user, the way of sending the prompt information to the user may, for example, include a pop-up window, and the prompt information may be presented in the form of text in the pop-up window. In addition, the pop-up window may also carry a select control for the user to choose to “agree” or “disagree” to provide the personal information to the electronic device.

It is to be understood that the above process of notifying and obtaining the user authorization is only illustrative and does not limit the implementations of the present disclosure. Other methods that satisfy relevant laws and regulations are also applicable to the implementations of the present disclosure.

### Example Environment

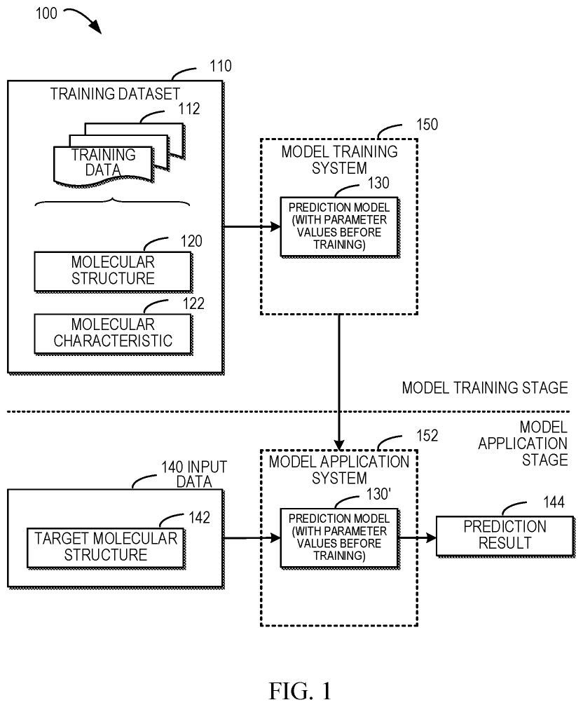

FIG. 1 illustrates a block diagram of an example environment 100 in which implementations of the present disclosure may be implemented. In the environment 100 of FIG. 1, it is desirable to train and use such a model (i.e., prediction model 130), which is configured to predict molecular characteristics with specific molecular structures (such as molecular force fields, molecular properties (such as solubility, stability, etc.), and so on. As shown in FIG. 1, the environment 100 includes a model training system 150 and a model application system 152. The upper part of FIG. 1 shows the process of the model training stage, and the lower part shows the process of the model application stage. Before training, parameter values of the prediction model 130 may have initial values or may have pretrained parameter values obtained through the pretraining process. After the training process, the parameter values of the prediction model 130 may be updated and adjusted. After training, a prediction model 130′ may be obtained. At this point, the parameter values of the prediction model 130′ have been updated, and based on the updated parameter values, the prediction model 130 may be used to achieve prediction tasks during the model application stage.

During the model training stage, the prediction model 130 may be trained using a model training system 150 based on a training dataset 110 that includes a plurality of training data 112. Here, each training data 112 may involve a two-tuples and include a molecular structure 120 and molecular characteristics 122. In the context of this disclosure, the molecular characteristics 122 may include molecular force fields, molecular properties (such as solubility, stability, etc.), and/or other characteristics in different training data 112.

At this point, the prediction model 130 may be trained using the training data 112 including the molecular structure 120 and the molecular characteristics 122. Specifically, the training process may be iteratively performed using a large amount of training data. After completion of the training, the prediction model 130 may determine the molecular characteristics associated with different molecular structures. In the model application stage, the prediction model 130′ (at this time, the prediction model 130′ has trained parameter values) may be called using the model application system 152. For example, input data 140 (including a target molecular structure 142) may be received, and a predicted result 144 of molecular characteristics of the target molecular structure 142 may be output.

In FIG. 1, the model training system 150 and the model application system 152 may include any computing system with computing power, such as various computing devices/systems, terminal devices, servers, etc. The terminal device may involve any type of mobile terminal, fixed terminal, or portable terminal, including a mobile phone, a desktop computer, a laptop, a notebook, a netbook, a tablet, a media computer, a multimedia tablet, or any combination thereof, including accessories and peripherals of these devices or any combination thereof. The server includes but is not limited to a mainframe, an edge computing node, a computing device in a cloud environment, etc.

It should be understood that the components and arrangements in the environment 100 shown in FIG. 1 are merely examples, and a computing system suitable for implementing the implementations described in this disclosure may include one or more different components, other components, and/or different arrangements. For example, although shown as separate, the model training system 150 and the model application system 152 may be integrated into the same system or device. The implementation of this disclosure is not limited in this regard. With reference to the accompanying drawings, implementations of the model training and model application will be described respectively.

It will be understood that the molecular characteristics 122 in the training data 112 should be consistent with a predicted target (i.e., the target expected to be output by the prediction model 130). In other words, when it is expected to predict the molecular force field, the molecular characteristics 122 in the training data 112 should be the measurement data of the molecular force field. At this time, the prediction model 130 may receive a molecular structure and output corresponding predicted values of the molecular force field. When it is expected to predict molecular properties (such as solubility), the molecular characteristics 122 in the training data 112 should be the measurement data of solubility. At this time, the prediction model 130 may receive a molecular structure and output corresponding predicted values of solubility.

In order to ensure prediction precision, it is necessary to collect a large amount of training data to train the prediction model 130. However, in most cases, there is only a small amount of training data, which may require lots of experiments. Furthermore, the field of molecular research involves millions (or even more) of commonly used molecular structures, which requires specialized experiments to be designed for respective molecular structure to obtain their molecular characteristics. At the same time, there are numerous prediction purposes in the field of molecular research, at which point training data has to be collected separately for these prediction purposes.

Currently, pretraining-finetuning technical solutions have been proposed, which focus on self-supervised learning policies. However, in molecular related prediction models, inputs (molecular structures) and outputs (molecular characteristics) have different inherent requirements for molecular modeling. Self-supervised learning tasks may only represent molecular structures, but lack intermediate knowledge to connect inputs and outputs. Self-learning pretraining may fill this gap to some extent, but due to the lack of large-scale labeled data, it may compromise the performance of downstream tasks.

In addition, supervised pretraining technical solutions have been proposed, which may perform multitask prediction on a large number of molecules based on molecular structures. However, the technical solution may lead to negative migration of downstream tasks, i.e., the prediction model obtained based on the technical solution is not “truly related” to downstream tasks, which results in unsatisfactory prediction precision. At this point, it is desirable to obtain more precise prediction models with limited training data for specific prediction purposes.

### Architecture of Molecular Prediction Model

In order to overcome the shortcomings of the foregoing technical solution, a two-stage training technical solution is proposed according to one implementation of the present disclosure. Specifically, the first stage is a pretraining process, which focuses on a basic physical characteristic (e.g., molecular energy) provided by a specific molecular structure and may first obtain a pretrained model. The second stage focuses on fine-tuning, that is, focusing on the association between the basic physical characteristic of molecules and other prediction purposes, at which point the pretrained model may be fine-tuned to obtain a higher-precision prediction model.

With the implementation of the present disclosure, a pretrained model may be generated based on a large amount of known public data during the pretraining stage. Afterwards, a molecular prediction model is established based on the pretrained model to achieve a specific prediction purpose, and the molecular prediction model is fine-tuned using a small amount of specialized training data for achieving that specific prediction 30) purpose. In this way, the precision of molecular prediction models may be improved with limited specialized training data.

With reference to FIG. 2, description is presented below to a summary according to one implementation of the present disclosure. FIG. 2 illustrates a block diagram 200 of a process for managing molecular prediction according to some implementations of the present disclosure. As shown in the figure, a pretrained model 240 may be first determined, which may describe the association between a molecular structure and molecular energy. The pretrained model 240 may include a plurality of network layers and may be utilized to generate a molecular prediction model 210 for a specific molecular prediction purpose 250. Here, the molecular prediction model 210 may include an upstream model 220 and a downstream model 230, and a portion of network layers 242 may be selected from the plurality of network layers of the pretrained model 240 to form the upstream model 220.

It will be understood that the molecular structure is based on spectroscopic data and used to describe the three-dimensional arrangement of atoms in molecules. It will be understood that the molecular structure is the inherent foundation of molecules and largely determines their other characteristics. Molecules with specific molecular structures will have similar characteristics, which are usually determined by the molecular energy. According to one implementation of the present disclosure, since the molecular structure and molecular energy are the foundation of other molecular related characteristics, it is proposed to utilize the pretrained model 240 (describing the association between the molecular structure and molecular energy) to construct a molecular prediction model 210 for achieving a specific prediction purpose.

At this point, the plurality of network layers of the pretrained model 240 have accumulated rich knowledge about intrinsic molecular factors, and the molecular prediction model 210 may be constructed directly using certain network layers from the plurality of network layers. In this way, training sample requirements for training the molecular prediction model 210 on zero basis may be greatly reduced, while maintaining the precision of the molecular prediction model 210. It will be understood that due to the existence of numerous publicly available molecular datasets, the pretrained model 240) may be generated using these datasets.

Furthermore, the downstream model 230 may be determined based on the specific molecular prediction purpose 250, and an output layer of the downstream model 230 is determined based on the molecular prediction purpose 250. Here, the molecular prediction purpose 250 represents a desired target to be output by the molecular prediction model 210. The molecular prediction model 210 may be generated based on the upstream model 220) and the downstream model 230 to describe the association between a molecular structure and the molecular prediction purpose 250 associated with the molecular structure. Here, the molecular prediction purpose 250) may represent the desired output target, such a as molecular force field, molecular properties, or other targets.

With the implementation of the present disclosure, on the one hand, the amount of specialized training data required to train the molecular prediction model 210 may be reduced, and on the other hand, the pretrained model 240 may be shared among different prediction purposes (such as molecular force fields, molecular properties, etc.), thereby improving the efficiency of generating the molecular prediction model 210.

### Model Training Process

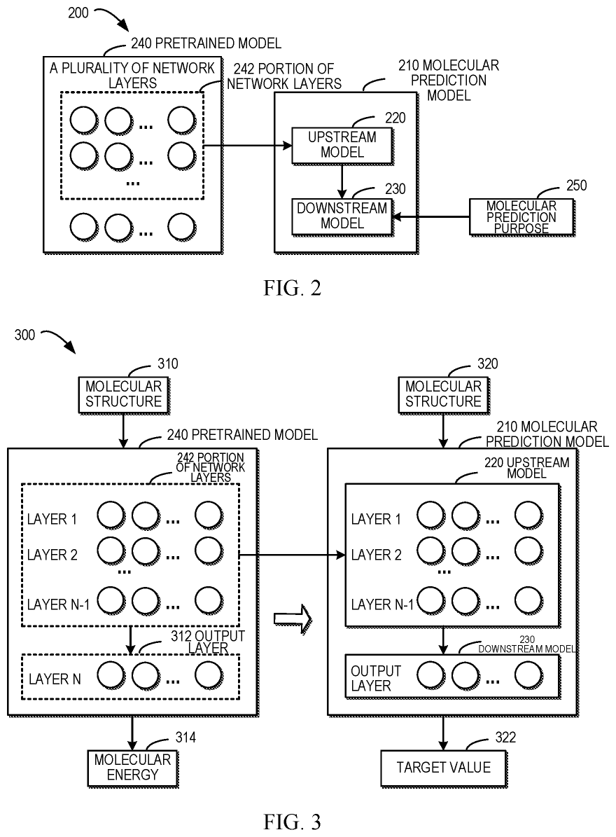

With reference to FIG. 3, description is presented below to more details about constructing the molecular prediction model 210 based on the pretrained model 240. FIG. 3 illustrates a block diagram 300 of a process for generating the molecular prediction model 210 based on the pretrained model 240 according to some implementations of the present disclosure. As shown in FIG. 3, the pretrained model 240 may describe the association between a molecular structure 310 and molecular energy 314. The pretrained model 240 may include N network layers, specifically, the first layer serves as an input layer for receiving the input molecular structure 310, and the Nth layer serves as an output layer 312 for outputting the molecular energy 314.

According to one implementation of the present disclosure, the upstream model 220 may be determined from a group of network layers other than the output layer 312 among a plurality of network layers in the pretrained model 240. For example, the first N−1 network layers in the pretrained model 240 may be directly used as the upstream model 220 of the molecular prediction model 210. Furthermore, the downstream model 230 may be generated based on the molecular prediction purpose 250. In this way, the molecular prediction model 210 may directly utilize the multifaceted knowledge about molecules obtained from the first to Nth, and then apply the knowledge to perform prediction tasks associated with the specific molecular prediction purpose 250. As shown in the figure, the molecular prediction model 210 may receive the molecular structure 320 and output a target value 322 corresponding to the molecular prediction purpose 250.

Hereinafter, more details about obtaining the pretrained model 240 will be described in detail. According to one implementation of the present disclosure, a backbone model for implementing the pretrained model 240 may be selected based on the molecular prediction purpose 250. For example, when the molecular prediction purpose 250 is to predict the molecular force field, the pretrained model 240 may be implemented based on the Geometric Message Passing Neural Network (GemNet) model. When the molecular prediction purpose 250 is to predict molecular properties, the pretrained model 240 may be implemented based on the E(n)-Equivariant Graph Neural Network (EGNN) model. Alternatively and/or additionally, any of the following models may be selected: Symmetric Gradient Domain Machine Learning (sGDML) model, NequIP model, GemNet-T model, and so on.

Alternatively and/or additionally, other numbers of network layers may be selected from the pretrained model 240, for example, the first to (N−2) th network layers may be selected, or fewer network layers may be selected. Although the number of selected network layers is relatively small at this time, the selected network layers still contain various knowledge about molecules. At this point, it is still possible to reduce the number of training samples required to train the molecular prediction model 210.

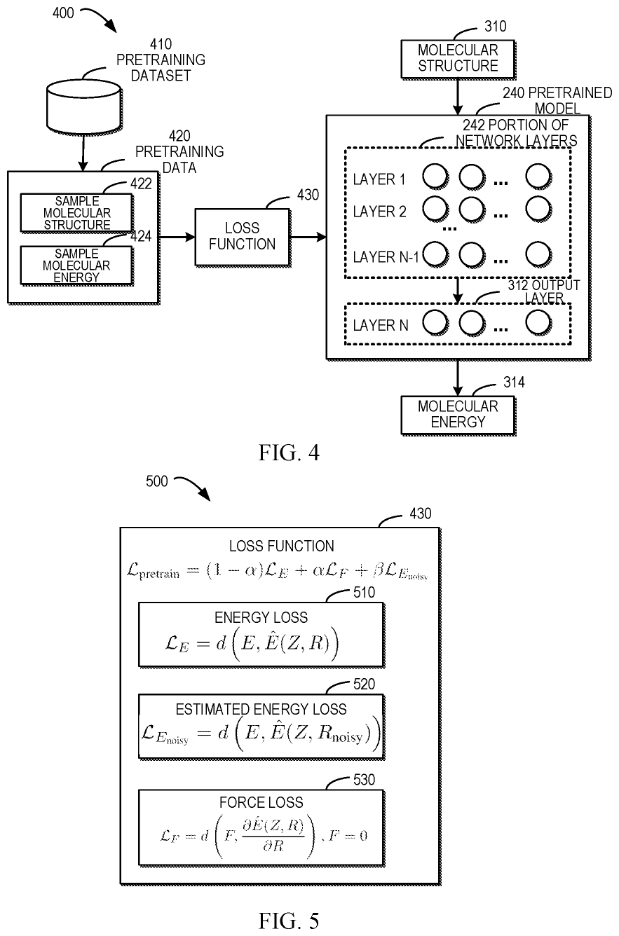

The training process performed on the pretrained model 240 may be referred to as a pretraining process, more detailed about which will be described with reference to FIG. 4. This figure illustrates a block diagram 400 of a process for obtaining the pretrained model 240) according to some implementations of the present disclosure. As shown in FIG. 4, the pretrained model may be trained using pretraining data 420 from a pretraining dataset 410, so that a loss function 430 associated with the pretrained model 240 meets a predetermined condition. The pretraining data 420 may include a sample molecular structure 422 and sample molecular energy 424.

It will be understood that research on molecular energy has been widely and extensively practiced for a long time, and a large number of publicly available datasets have been provided so far. For example, the PubChemQC PM6 dataset is a publicly available dataset that includes billions of molecular structures and their corresponding electronic characteristics. For another example, the Quantum Machine 9 (QM9) dataset provides information on the geometric structure, energy, electronic, and thermodynamic characteristics of molecules. These publicly available datasets (or a portion thereof) may be used as training data to obtain the pretrained model 240. In other words, after the training process, the specific configuration of the first to Nth network layers in the pretrained model 240 may be obtained.

As shown in FIG. 4, the pretraining dataset 410 may include a plurality of training data 420, and the training data 420 may include a sample molecular structure 422 and sample molecular energy 424. Description is presented below on how to perform the pretraining process by taking the PubChemQC PM6 dataset as a specific example of the pretraining dataset 410. The PubChemQC PM6 dataset includes a large number of molecular structures and their corresponding electronic characteristics. For example, this dataset includes approximately 86 million optimized 3D molecular structures and their associated molecular energy. These molecular structures and energy may be utilized as training data. Specifically, the backbone model of the pretrained model 240 may be selected, and the loss function 430 of the pretrained model 240 may be constructed. The loss function 430 may represent the difference between the true and predicted values of the sample data, thereby allowing the pretraining process to iteratively optimize the pretrained model 240 in a direction that gradually reduces the difference.

With the implementation of the present disclosure, various publicly available datasets may be directly used as the pretraining datasets 410. On the one hand, these publicly available datasets include a huge amount of sample data, so that fundamental knowledge of molecular structures and molecular energy may be obtained without the need for specialized training data. On the other hand, the sample data in these datasets have been studied for a long time and have been proven to be accurate or relatively accurate, so that a more accurate pretrained model 240 may be obtained by performing the pretraining process based on the sample data. Furthermore, since the molecular prediction model 210 that achieves the specific molecular prediction purpose 250 includes a portion of the pretrained model 240, it may be ensured that the subsequent generated molecular prediction model 210 is also reliable.

According to one implementation of the present disclosure, the loss function 430 may include multiple aspects of content, and FIG. 5 illustrates a block diagram 500 of the loss function 430 for the pretraining model 240 according to some implementations of the present disclosure. As shown in FIG. 5, the loss function 430 may include an energy loss 510, where the energy loss 510 represents the difference between the sample molecular energy 424 and the predicted value of the sample molecular energy 424 obtained based on the sample molecular structure 422. Specifically, the energy loss 510 may be determined based on the following Formula 1.

$${ℒ}_{E} = d ( E , \hat{E} ( Z , R ) ) \qquad Formula 1$$

In formula 1, the symbol [symbol] _{E} represents the energy loss 510, the symbol R represents a molecular structure, the symbol E represents molecular energy of molecules with the molecular structure R, Ê (Z, R) represents the predicted value of the molecular energy E obtained based on the molecular structure R and the pretrained model 240, and d represents the difference between E and Ê. According to one implementation of the present disclosure, molecular structures may be described in different formats. For example, molecular structures may be represented in SMILES or other formats: For another example, molecular structures in atomic coordinate form may be further obtained through tools such as RDKIT: For further example, molecular structures may be represented in the form of molecular diagrams.

With the implementation of the present disclosure, Formula 1 may quantitatively represent the pretrained target. In this way, based on the respective pretraining data 420 in the pretraining dataset 410, the parameters of respective network layers of the pretraining model 240 may be adjusted in a way that minimizes the energy loss 510, so that the pretraining model 240 may accurately describe the association between the molecular structure 310 and the molecular energy 314.

It will be understood that the training dataset for downstream prediction tasks typically only provides molecular structures in SMILES format and does not provide precise atomic coordinates. At this point, the loss function 430 may include an estimated energy loss 520, which represents the difference between the sample molecular energy 424 and the predicted value of the sample molecular energy 424 obtained based on the sample molecular structure 422, where the sample molecular structure is estimated. Specifically, the estimated energy loss 520 may be determined based on the following Formula 2.

$${ℒ}_{{E}_{n o i s y}} = d ( E , \hat{E} ( Z , {R}_{noisy} ) ) \qquad Formula 2$$

In Formula 2, the symbol [symbol] _{E}_{_{noisy}} represents the estimated energy loss 520, the symbol R_{noisy} represents the estimated molecular structure, the symbol E represents the molecular energy of molecules with the molecular structure R_{noisy}, Z represents the pretrained model 240, Ê(Z, R_{noisy}) represents the predicted value of the molecular energy E obtained based on the estimated molecular structure R_{noisy} and the pretrained model 240, and d represents the difference between E and E. At this point, the estimated molecular structure may be determined from SMILES using tools such as RDKIT. With the implementation of the present disclosure, Formula 2 may quantitatively represent the pretrained target. At this point, the expression of the estimated molecular structure R_{noisy} is consistent with the input molecular structure of the downstream task, which may improve the accuracy of the prediction results.

Alternative and/or additionally, data augmentation may be further provided during the pretraining process, i.e., additional loss functions are determined based on existing data in the training dataset 410. Specifically, the loss function 430 may include a force loss 530, which represents the difference between a predetermined gradient (e.g., 0) and a gradient of the predicted value of the sample molecular energy 424 obtained based on the sample molecular structure 422 relative to the sample molecular structure 422. It will be understood that the PubChemQC PM6 dataset is established for the purpose of molecular optimization geometry, thus the molecular energy may be minimized. Molecular force represents the gradient of energy relative to atomic coordinates, and since the molecule is relatively stable at this time, the gradient should have a value close to 0. At this point, data augmentation may be achieved based on the pretraining data 420 in the pretraining dataset 410, that is, the potential force applied to atoms is a gradient of energy. This is equivalent to assuming a supervised learning loss for the force with a label of 0. That is to say, the force loss 530 may be determined based on the following Formula 3.

$${ℒ}_{F} = d ( F , \frac{\partial \hat{E} ( Z , R )}{\partial R} ) , F = 0 \qquad Formula 3$$

In Formula 3, [symbol] _{F} represents the force loss 530,

$$\frac{\partial \hat{E} ( Z , R )}{\partial R}$$

represents the gradient of the predicted value Ê(Z, R) of the molecular energy obtained based on the molecular structure R and the pretrained model Z relative to the molecular structure, F represents the predetermined gradient (F=0), and

$$d ( F , \frac{\partial \hat{E} ( Z ,}{\partial R} )$$

represents the difference between the calculated gradient and the predetermined gradient F=0. With the implementation of the present disclosure, data augmentation may be performed on the pretraining dataset 410 to include more knowledge about molecular forces in the pretrained model 240. In this way, the precision of the pretrained model 240 may be improved, thereby providing more accurate prediction results when the molecular prediction purpose 250 relates to molecular force fields.

According to one implementation of the present disclosure, the loss function 430 may be determined based on any of Formulas 1 to 3. Furthermore, two or more of Formulas 1 to 3 may be comprehensively considered. For example, the loss function 430 for pretraining may be determined based on any of the following Formulas 4 to 7.

$${ℒ}_{p r etrαin} = ( 1 - α ) {ℒ}_{E} + {αℒ}_{F} \qquad Formula 4$$

$${ℒ}_{p r etrαin} = ( 1 - α ) {ℒ}_{E} + {βℒ}_{{E}_{n o i s y}} \qquad Formula 5$$

$${ℒ}_{p r etrαin} = {αℒ}_{F} + {βℒ}_{{E}_{n o i s y}} \qquad Formula 6$$

$${ℒ}_{p r etrαin} = ( 1 - α ) {ℒ}_{E} + {αℒ}_{F} + {βℒ}_{{E}_{n o i s y}} \qquad Formula 7$$

In Formulas 4 to 7, the meanings of respective symbols are the same as those described in the previous formula, and a and B respectively represent a predetermined value between [0,1]. According to one implementation of the present disclosure, the loss function 430 may be determined based on specific prediction purposes. For example, when it is desirable to predict the molecular force field, Formulas 3, 4, 6, or 7 may be used. When downstream data involves estimated molecular structures, Formulas 2, 5, 6, or 7 may be used, and so on.

According to one implementation of the present disclosure, a predetermined stop condition may be specified to stop the pretraining process when the pretrained model 240 meets this stop condition. With the implementation of the present disclosure, complex pretraining processes may be converted into simple mathematical operations based on Formulas 1 to 7. In this way, a higher-precision pretrained model 240 may be obtained using a publicly available training dataset 610 without the need to prepare dedicated training data.

The specific process of pretraining has been described above. After obtaining the pretrained model 240, the first to (N−1) th network layers in the pretrained model 240 may be directly used as the upstream model 220 of the molecular prediction model 210. Furthermore, the downstream model 230 of the molecular prediction model 210 may be determined based on the molecular prediction purpose 250. Specifically, the downstream model 230 may include one or more network layers. According to one implementation of the present disclosure, the molecular prediction purpose 250 may include a molecular force field and/or molecular properties. At this point, the downstream model 230 may be implemented using a single network layer, i.e., the downstream model 230 only includes a single output layer. Alternatively and/or additionally, the downstream model 230 may also include two or more network layers. At this point, the last network layer among a plurality of network layers in the downstream model 230 is the output layer of the downstream model 230.

According to one implementation of the present disclosure, the upstream model 220) and the downstream model 230 may be connected to obtain the final molecular prediction model 210. It will be understood that the parameters in the upstream model 220 are directly obtained from the pretrained model 240, and the parameters of the downstream model 230 may be set to any initial values and/or numerical values obtained by other means. According to one implementation of the present disclosure, random initial values may be used. Downstream tasks may require the final output layer to have outputs of different dimensions than pretraining, or even if the dimensions are the same, since less bias loss gradients are provided during fine-tuning, randomly initializing the parameters of the output layer may usually achieve the higher-precision molecular prediction model 210.

Subsequently, the molecular prediction model 210 may be used as the overall prediction model and trained using a dedicated dataset associated with the molecular prediction purpose 250. With the implementation of the present disclosure, since the upstream model 220 already includes various knowledge about molecules, a higher-precision molecular prediction model 210 may be obtained with a small amount of specialized training data.

Furthermore, more details on training the molecular prediction model 210 will be described with reference to FIG. 6. As shown in FIG. 6, the molecular prediction model 210 may be trained using training data 620 in the training dataset 610, so that the loss function 630 associated with the molecular prediction model 210 meets a predetermined condition. Here, the training data 620 may include a sample molecular structure 622 and a sample target measurement value 624 corresponding to the molecular prediction purpose 250. Specifically, assuming that the molecular prediction purpose 250 is a molecular force field, the sample target measurement value 624 may be a measurement value of the molecular force field: Assuming that the molecular prediction purpose 250 is soluble, the sample target measurement value 624 may be a soluble measurement value.

According to one implementation of the present disclosure, the training dataset 610 corresponding to the molecular prediction purpose 250 may be obtained, which may be a specialized dataset prepared for the molecular prediction purpose 250 (e.g., through experiments, etc.). Compared with the pretraining dataset 410 that includes a large amount of pretraining data (e.g., millions or even more), the training dataset 610 typically includes less training data (e.g., thousands or even fewer). In this way, the higher-precision molecular prediction model 210 may be obtained without the no need to collect the massive specialized training data but using limited specialized training data.

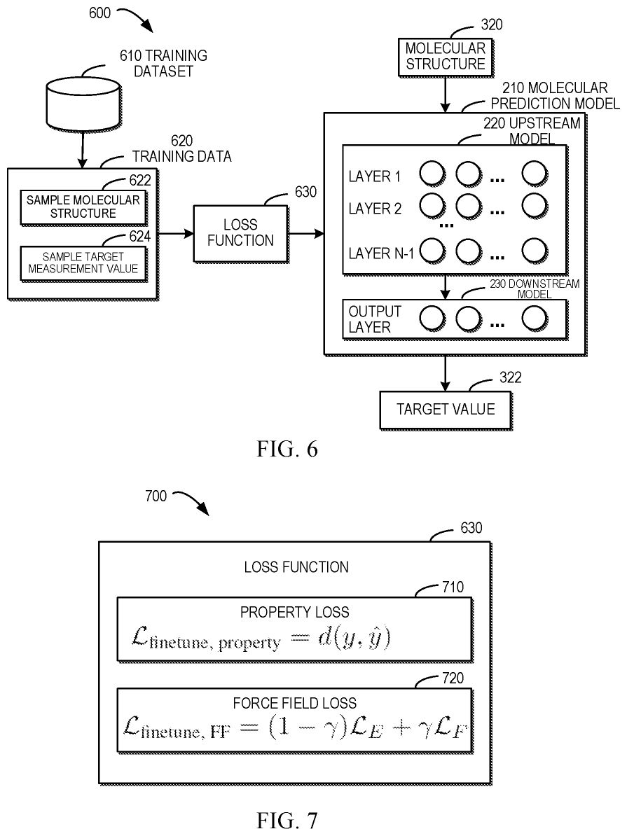

According to one implementation of the present disclosure, the loss function 630 may be constructed for the molecular prediction model 210. FIG. 7 illustrates a block diagram 700 of the loss function 630 used for the molecular prediction model 210 according to some implementations of the present disclosure. As shown in FIG. 7, the loss function 630 of the molecular prediction model 210 may include an energy loss 710, which includes the difference between the sample target measurement value 624 and the predicted value of the sample target measurement value 624 obtained based on the sample molecular structure 622.

When it is desirable to predict molecular properties, the energy loss 710 may be determined based on the following Formula 8.

$${ℒ}_{finetune , property} = d ( y , \hat{y} ) \qquad Formula 8$$

In Formula 8, [symbol] _{finetune, property} represents the property loss 710 of the molecular prediction model 210, y represents the sample target measurement value 624 (corresponding to the molecular structure R) in the training data 620, and ŷ represents the predicted value obtained based on the molecular structure R and the molecular prediction model 210, and d (y, ŷ) represents the difference between y and ŷ. In this way, the loss function 630 may be determined by Formula 8, and fine-tuning may be performed in the direction that minimizes the loss function 630. In this way, the complex process of fine-tuning the molecular prediction model 210 may be converted into simple and effective mathematical operations.

According to one implementation of the present disclosure, when it is desirable to predict a molecular force field, the loss function 630 of the molecular prediction model 210 may further include a force field loss 720. The force field loss 720 includes the difference between a predetermined gradient and a gradient of the predicted value of the sample molecular energy 624 obtained based on the sample molecular structure 622 relative to the sample molecular structure 622. Specifically, the force field loss 720 may be determined based on the following Formula 9.

$${ℒ}_{finetune , FF} = ( 1 - γ ) {ℒ}_{E} + {γℒ}_{F} \qquad Formula 9$$

In Formula 8, [symbol] _{finetune, FF} represents the force field loss 720 of the molecular prediction model 210, and the meanings of respective symbols are the same as those described in the previous formula, and y represents a predetermined value between [0,1]. In this way, the loss function may be determined by Formula 0, and further the complex process of fine-tuning the molecular prediction model 210 may be converted into simple and effective mathematical operations. With the implementation of the present disclosure, the molecular prediction model 210 may be obtained in a more accurate and effective manner.

The process for obtaining the molecular prediction model 210 has been described with reference to the figures above. With the implementation of the present disclosure, the pretrained model 240 may be trained based on a large amount of known and publicly available data. Furthermore, the molecular prediction model 210 may be further fine-tuned based on a smaller specialized training dataset that includes a limited number of training data. In this way, an effective balance may be achieved between the training accuracy and various costs of preparing a large amount of specialized training data, thereby obtaining a higher-precision molecular prediction model 210 at a lower cost.

### Model Application Process

While the training of the molecular prediction model 210 has been described above, description will be presented below on how to determine the predicted values associated with the molecular prediction purpose 250 by using the molecular prediction model 210. According to one implementation of the present disclosure, after completing the model training stage, received input data may be processed using the trained molecular prediction model 210 with trained parameter values. If a target molecular structure is received, the predicted value corresponding to the molecular prediction purpose may be determined based on the molecular prediction model 210.

For example, the target molecular structure to be processed may be input into the molecular prediction model 210. At this point, the target molecular structure may be represented based on SMILES format or atomic coordinate form. The molecular prediction model 210 may output the predicted values corresponding to the template molecular structure. Here, depending on the molecular prediction purpose 250, the predicted value may include a predicted value of the corresponding target. Specifically, when the molecular prediction model 210 is used to predict the molecular force field, the molecular prediction model 210 may output the predicted value of the molecular force field. In this way, the trained molecular prediction model 210 may have higher precision, providing a judgment basis for subsequent processing operations.

According to one implementation of the present disclosure, in the application environment of predicting molecular force fields, the prediction results of the molecular prediction model 210 have achieved higher precision in both in-domain testing and out-of-domain testing. For example, Table 1 below shows the in-domain test data.

TABLE 1
In-Domain Test Data

| | Model | | | | |
|---|---|---|---|---|---|
| | | | | Improved | Relative |
| Molecule | SGDML | NequIP | GemNet-T | GemNet-T | improvement |
| Aspirin | 33.0 | 14.7 | 12.6 | 10.2 | 19% |
| Benzene | 1.7 | 0.8 | 1.9 | 1.3 | 31.6% |
| Ethanol | 15.2 | 9.4 | 4.0 | 4.0 | 1.8% |
| Propylene | 16.0 | 16.0 | 6.5 | 6.2 | 4.6% |
| glycol | | | | | |
| Toluene | 9.1 | 4.4 | 4.0 | 3.6 | 10% |

In Table 1, the rows represent the backbone models on which different prediction models are based, and the columns represent the error data of the predicted values related to the molecular force field based on different prediction models. Specifically, the data in the second row “Aspirin” indicate that the correlation error of predicting the molecular force field of aspirin using the sGDML model is 33.0, the correlation error data using the NequIP model is 14.7, the correlation error data using the GemNet-T model is 12.6, and the correlation error data using the improved GemNet-T model based on the method of the present disclosure is 10.2. It may be seen that the relative improvement has reached 19.0%. Similarly, the other columns in Table 1 show the relevant data for predicting molecular force fields for other molecules. It may be seen from Table 1 that with the implementation of the present disclosure, the error of molecular force field prediction may be greatly reduced, and higher accuracy may be provided. Furthermore, the improved GemNet-T also has achieved high accuracy in out-of-domain testing.

According to one implementation of the present disclosure, in an application environment for predicting molecular properties, the molecular prediction model 210 may output soluble predicted values. The EGNN model may be improved using the method of the present disclosure, so as to predict molecular properties. At this point, the improved EGNN model achieves better prediction performance. It will be understood that although solubility is used as an example of molecular properties above, the molecular properties here may include various molecular properties, such as solubility, stability, reactivity, polarity, phase state, color, magnetism, and biological activity, etc. With the implementation of the present disclosure, an accurate and reliable molecular prediction model 210 may be obtained with less dedicated training data, and molecular properties may be predicted using the molecular prediction model 210.

### Example Process

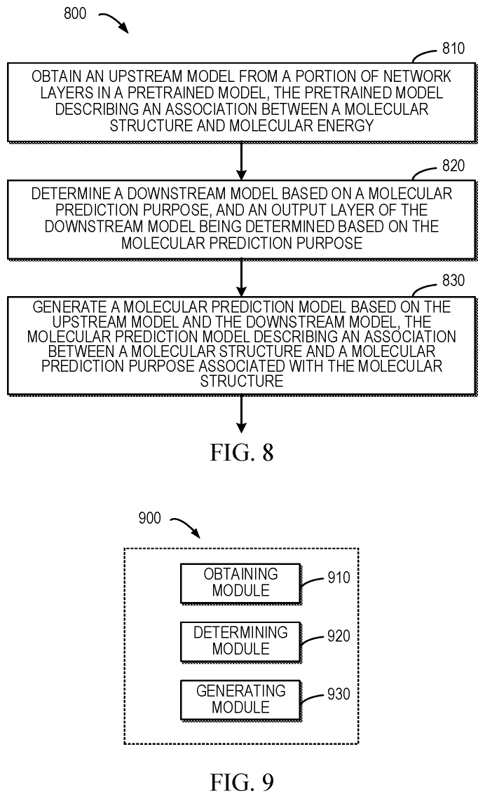

FIG. 8 illustrates a flowchart of a method 800 for managing molecular prediction according to some implementations of the present disclosure. Specifically, at block 810, an upstream model is obtained from a portion of network layers in a pretrained model, the pretrained model describing an association between a molecular structure and molecular energy; At block 820, a downstream model is determined based on a molecular prediction purpose, and an output layer of the downstream model is determined based on the molecular prediction purpose: And at block 830, a molecular prediction model is generated based on the upstream model and the downstream model, the molecular prediction model describing an association between a molecular structure and a molecular prediction purpose associated with the molecular structure.

According to one implementation of the present disclosure, obtaining the upstream model comprises: obtaining the pretrained model, which comprises a plurality of network layers; and selecting the upstream model from a group of network layers other than an output layer of the pretrained model from the plurality of network layers.

According to one implementation of the present disclosure, obtaining the pretrained model comprises: training the pretrained model using pretraining data in a pretraining dataset, such that a loss function associated with the pretrained model satisfies a predetermined condition, the pretraining data comprising a sample molecular structure and sample molecular energy.

According to one implementation of the present disclosure, the loss function comprises at least any of: energy loss, the energy loss representing the difference between the sample molecular energy and a predicted value of the sample molecular energy based on the sample molecular structure: estimated energy loss, the estimated energy loss representing the difference between the sample molecular energy and a predicted value of the sample molecular energy based on the sample molecular structure, the sample molecular structure being estimated; and force loss, the force loss representing the difference between a predetermined gradient and a gradient of a predicted value of the sample molecular energy obtained based on the sample molecular structure relative to the sample molecular structure.

According to one implementation of the present disclosure, the molecular prediction purpose comprises at least any of: molecular properties and molecular force fields, and the pretrained model is selected based on the molecular prediction purpose.

According to one implementation of the present disclosure, the downstream model comprises at least one downstream network layer, and the last downstream network layer in the at least one downstream network layer is the output layer of the downstream model.

According to one implementation of the present disclosure, generating the molecular prediction model based on the upstream model and the downstream model comprises: connecting the upstream model and the downstream model to form the molecular prediction model; and training the molecular prediction model using training data in a training dataset, such that a loss function of the molecular prediction model satisfies a predetermined condition, the training data comprising a sample molecular structure and a sample target measurement value corresponding to the molecular prediction purpose.

According to one implementation of the present disclosure, the loss function of the molecular prediction model comprises the difference between the sample target measurement value and a predicted value of the sample target measurement value obtained based on the sample molecular structure.

According to one implementation of the present disclosure, in response to determining the molecular force field as the molecular prediction purpose, the loss function of the molecular prediction model further comprises: the difference between a predetermined gradient and a gradient of a predicted value of the sample molecular energy obtained based on the sample molecular structure relative to the sample molecular structure.

According to one implementation of the present disclosure, the method 800 further comprises: in response to receiving a target molecular structure, determining a predicted value corresponding to the molecular prediction purpose based on the molecular prediction model.

### Example Apparatus and Device

FIG. 9 illustrates a block diagram of an apparatus 900 for managing molecular prediction according to some implementations of the present disclosure. The apparatus 900 comprises: an obtaining module 910 configured for obtaining an upstream model from a portion of network layers in a pretrained model, the pretrained model describing an association between a molecular structure and molecular energy: a determining module 920 configured for determining a downstream model based on a molecular prediction purpose, and an output layer of the downstream model being determined based on the molecular prediction purpose; and a generating module 930 configured for generating a molecular prediction model based on the upstream model and the downstream model, the molecular prediction model describing an association between a molecular structure and a molecular prediction purpose associated with the molecular structure.

According to one implementation of the present disclosure, the obtaining module 910 comprises: a pre-obtaining module configured for obtaining the pretrained model, which comprises a plurality of network layers; and a selecting module configured for selecting the upstream model from a group of network layers other than an output layer of the pretrained model from the plurality of network layers.

According to one implementation of the present disclosure, the pre-obtaining module comprises: a pre-training module configured for training the pretrained model using pretraining data in a pretraining dataset, such that a loss function associated with the pretrained model satisfies a predetermined condition, the pretraining data comprising a sample molecular structure and sample molecular energy.

According to one implementation of the present disclosure, the loss function comprises at least any of: energy loss, the energy loss representing the difference between the sample molecular energy and a predicted value of the sample molecular energy based on the sample molecular structure: estimated energy loss, the estimated energy loss representing the difference between the sample molecular energy and a predicted value of the sample molecular energy based on the sample molecular structure, the sample molecular structure being estimated; and force loss, the force loss representing the difference between a predetermined gradient and a gradient of a predicted value of the sample molecular energy obtained based on the sample molecular structure relative to the sample molecular structure.

According to one implementation of the present disclosure, the molecular prediction purpose comprises at least any of: molecular properties and molecular force fields, and the pretrained model is selected based on the molecular prediction purpose.

According to one implementation of the present disclosure, the downstream model comprises at least one downstream network layer, and the last downstream network layer in the at least one downstream network layer is the output layer of the downstream model.

According to one implementation of the present disclosure, the generating module 930 comprises: a connecting module configured for connecting the upstream model and the downstream model to form the molecular prediction model; and a training module configured for training the molecular prediction model using training data in a training dataset, such that a loss function of the molecular prediction model satisfies a predetermined condition, the training data comprising a sample molecular structure and a sample target measurement value corresponding to the molecular prediction purpose.

According to one implementation of the present disclosure, the loss function of the molecular prediction model comprises the difference between the sample target measurement value and a predicted value of the sample target measurement value obtained based on the sample molecular structure.

According to one implementation of the present disclosure, in response to determining the molecular force field as the molecular prediction purpose, the loss function of the molecular prediction model further comprises: the difference between a predetermined gradient and a gradient of a predicted value of the sample molecular energy obtained based on the sample molecular structure relative to the sample molecular structure.

According to one implementation of the present disclosure, the apparatus 900 further comprises: a predicted value determining module configured for, in response to receiving a target molecular structure, determining a predicted value corresponding to the molecular prediction purpose based on the molecular prediction model.

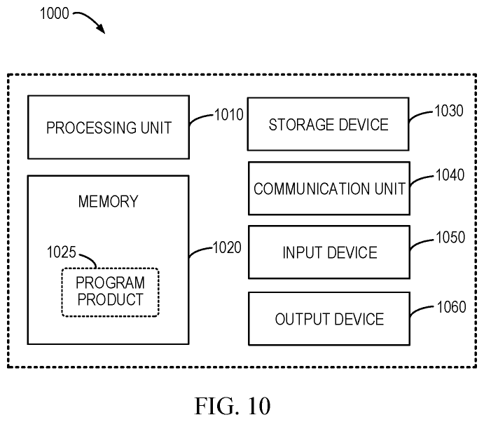

FIG. 10 illustrates a block diagram of a device 100 that may implement a plurality of implementations of the present disclosure. It should be understood that the computing device 100 shown in FIG. 10 is only exemplary and shall not constitute any limitation on the functions and scopes of the implementations described herein. The computing device 1000 shown in FIG. 10 may be used to implement the method 600 as shown in FIG. 6.

As shown in FIG. 10, the computing device 100 is in the form of a general purpose computing device. Components of the computing device 100 may include, but are not limited to, one or more processors or processing units 1010, a memory 1020, a storage device 1030, one or more communication units 1040, one or more input devices 1050, and one or more output devices 1060. The processing unit 1010 may be a physical or virtual processor and may execute various processing based on the programs stored in the memory 1020. In a multi-processor system, a plurality of processing units executes computer-executable instructions in parallel to enhance parallel processing capability of the computing device 1000.

The computing device 100 usually includes a plurality of computer storage mediums. Such mediums may be any attainable medium accessible by the computing device 1000, including but not limited to, a volatile and non-volatile medium, a removable and non-removable medium. The memory 1020 may be a volatile memory (e.g., a register, a cache, a Random Access Memory (RAM)), a non-volatile memory (such as, a Read-Only Memory (ROM), an Electrically Erasable Programmable Read-Only Memory (EEPROM), flash), or any combination thereof. The storage device 1030 may be a removable or non-removable medium, and may include a machine-readable medium (e.g., a memory, a flash drive, a magnetic disk) or any other medium, which may be used for storing information and/or data (e.g., training data for training) and be accessed within the computing device 1000.

The computing device 1000 may further include additional removable/non-removable, volatile/non-volatile storage mediums. Although not shown in FIG. 10, there may be provided a disk drive for reading from or writing into a removable and non-volatile disk (e.g., “floppy disk”) and an optical disc drive for reading from or writing into a removable and non-volatile optical disc. In such cases, each drive may be connected to a bus (not shown) via one or more data medium interfaces. The memory 1020 may include a computer program product 1025 having one or more program modules, and these program modules are configured for performing various methods or acts of various implementations of the present disclosure.

The communication unit 1040 implements communication with another computing device via a communication medium. Additionally, functions of components of the computing device 1000 may be realized by a single computing cluster or a plurality of computing machines, and these computing machines may communicate through communication connections. Therefore, the computing device 1000 may operate in a networked environment using a logic connection to one or more other servers, a Personal Computer (PC) or a further general network node.

The input device 1050 may be one or more various input devices, such as a mouse, a keyboard, a trackball, a voice-input device, and the like. The output device 1060 may be one or more output devices, e.g., a display, a loudspeaker, a printer, and so on. The computing device 1000 may also communicate through the communication unit 1040 with one or more external devices (not shown) as required, where the external device, e.g., a storage device, a display device, and so on, communicates with one or more devices that enable users to interact with the computing device 1000, or with any device (such as a network card, a modem, and the like) that enable the computing device 1000 to communicate with one or more other computing devices. Such communication may be executed via an Input/Output (I/O) interface (not shown).

According to the implementations of the present disclosure, a computer-readable storage medium is provided, on which computer-executable instructions are stored, wherein the computer-executable instructions are executed by a processor to implement the method described above. According to the implementations of the present disclosure, a computer program product is further provided, which is tangibly stored on a non-transient computer-readable medium and includes computer-executable instructions, which are executed by a processor to implement the method described above. According to the implementations of the present disclosure, a computer program product is provided, storing a computer program thereon, the program, when executed by a processor, implementing the method described above.

Aspects of the present disclosure are described herein with reference to flowchart illustrations and/or block diagrams of methods, apparatus (systems), and computer program products according to implementations of the invention. It will be understood that each block of the flowchart illustrations and/or block diagrams, and combinations of blocks in the flowchart illustrations and/or block diagrams, may be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that may direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods and computer program products according to various implementations of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, may be implemented by special purpose hardware-based systems that perform the specified functions or acts, or combinations of special purpose hardware and computer instructions.

The descriptions of the various implementations of the present disclosure have been presented for purposes of illustration, but are not intended to be exhaustive or limited to implementations disclosed. Many modifications and variations will be apparent to those of ordinary skill in the art without departing from the scope and spirit of the described implementations. The terminology used herein was chosen to best explain the principles of implementations, the practical application or technical improvement over technologies found in the marketplace, or to enable others of ordinary skill in the art to understand implementations disclosed herein.

1. A method for managing molecular prediction, comprising:
obtaining an upstream model from a portion of network layers in a pretrained model, the pretrained model describing an association between a molecular structure and molecular energy;
determining a downstream model based on a molecular prediction purpose, and an output layer of the downstream model being determined based on the molecular prediction purpose; and
generating a molecular prediction model based on the upstream model and the downstream model, the molecular prediction model describing an association between a molecular structure and a molecular prediction purpose associated with the molecular structure.

2. The method of claim 1, wherein obtaining the upstream model comprises:
obtaining the pretrained model, which comprises a plurality of network layers; and
selecting the upstream model from a group of network layers other than an output layer of the pretrained model from the plurality of network layers.

3. The method of claim 1, wherein obtaining the pretrained model comprises:
training the pretrained model using pretraining data in a pretraining dataset, such that a loss function associated with the pretrained model satisfies a predetermined condition, the pretraining data comprising a sample molecular structure and sample molecular energy.

4. The method of claim 3, wherein the loss function comprises at least any of:
energy loss, the energy loss representing a difference between the sample molecular energy and a predicted value of the sample molecular energy based on the sample molecular structure;
estimated energy loss, the estimated energy loss representing a difference between the sample molecular energy and a predicted value of the sample molecular energy based on the sample molecular structure, the sample molecular structure being estimated; and
force loss, the force loss representing a difference between a predetermined gradient and a gradient of a predicted value of the sample molecular energy obtained based on the sample molecular structure relative to the sample molecular structure.

5. The method of claim 1, wherein the molecular prediction purpose comprises at least any of: a molecular property and a molecular force field, and the pretrained model is selected based on the molecular prediction purpose.

6. The method of claim 5, wherein the downstream model comprises at least one downstream network layer, and the last downstream network layer in the at least one downstream network layer is the output layer of the downstream model.

7. The method of claim 5, wherein generating the molecular prediction model based on the upstream model and the downstream model comprises:
connecting the upstream model and the downstream model to form the molecular prediction model; and
training the molecular prediction model using training data in a training dataset, such that a loss function of the molecular prediction model satisfies a predetermined condition, the training data comprising a sample molecular structure and a sample target measurement value corresponding to the molecular prediction purpose.

8. The method of claim 7, wherein the loss function of the molecular prediction model comprises the difference between the sample target measurement value and a predicted value of the sample target measurement value obtained based on the sample molecular structure.

9. The method of claim 8, wherein in response to determining the molecular force field as the molecular prediction purpose, the loss function of the molecular prediction model further comprises: a difference between a predetermined gradient and a gradient of a predicted value of the sample molecular energy obtained based on the sample molecular structure relative to the sample molecular structure.

10. The method of claim 1, further comprising: in response to receiving a target molecular structure, determining a predicted value corresponding to the molecular prediction purpose based on the molecular prediction model.

11-18. (canceled)

19. An electronic device, comprising:
at least one processing unit; and
at least one memory coupled to the at least one processing unit and storing instructions executed by the at least one processing unit, the instructions, when executed by the at least one processing unit, causing the electronic device to perform a method for managing molecular prediction, the method comprising:
obtaining an upstream model from a portion of network layers in a pretrained model, the pretrained model describing an association between a molecular structure and molecular energy;
determining a downstream model based on a molecular prediction purpose, and an output layer of the downstream model being determined based on the molecular prediction purpose; and
generating a molecular prediction model based on the upstream model and the downstream model, the molecular prediction model describing an association between a molecular structure and a molecular prediction purpose associated with the molecular structure.

20. A non-transitory computer-readable storage medium, storing a computer program thereon, the computer program, when executed by a processor, causing the processor to implement a method for managing molecular prediction, the method comprising:
obtaining an upstream model from a portion of network layers in a pretrained model, the pretrained model describing an association between a molecular structure and molecular energy;
determining a downstream model based on a molecular prediction purpose, and an output layer of the downstream model being determined based on the molecular prediction purpose; and
generating a molecular prediction model based on the upstream model and the downstream model, the molecular prediction model describing an association between a molecular structure and a molecular prediction purpose associated with the molecular structure.

21. The device of claim 19, wherein obtaining the upstream model comprises:
obtaining the pretrained model, which comprises a plurality of network layers; and
selecting the upstream model from a group of network layers other than an output layer of the pretrained model from the plurality of network layers.

22. The device of claim 19, wherein obtaining the pretrained model comprises:
training the pretrained model using pretraining data in a pretraining dataset, such that a loss function associated with the pretrained model satisfies a predetermined condition, the pretraining data comprising a sample molecular structure and sample molecular energy.

23. The device of claim 22, wherein the loss function comprises at least any of:
energy loss, the energy loss representing a difference between the sample molecular energy and a predicted value of the sample molecular energy based on the sample molecular structure;
estimated energy loss, the estimated energy loss representing a difference between the sample molecular energy and a predicted value of the sample molecular energy based on the sample molecular structure, the sample molecular structure being estimated; and
force loss, the force loss representing a difference between a predetermined gradient and a gradient of a predicted value of the sample molecular energy obtained based on the sample molecular structure relative to the sample molecular structure.

24. The device of claim 19, wherein the molecular prediction purpose comprises at least any of: a molecular property and a molecular force field, and the pretrained model is selected based on the molecular prediction purpose.

25. The device of claim 24, wherein the downstream model comprises at least one downstream network layer, and the last downstream network layer in the at least one downstream network layer is the output layer of the downstream model.

26. The device of claim 24, wherein generating the molecular prediction model based on the upstream model and the downstream model comprises:
connecting the upstream model and the downstream model to form the molecular prediction model; and
training the molecular prediction model using training data in a training dataset, such that a loss function of the molecular prediction model satisfies a predetermined condition, the training data comprising a sample molecular structure and a sample target measurement value corresponding to the molecular prediction purpose.

27. The device of claim 26, wherein the loss function of the molecular prediction model comprises the difference between the sample target measurement value and a predicted value of the sample target measurement value obtained based on the sample molecular structure.

28. The device of claim 27, wherein in response to determining the molecular force field as the molecular prediction purpose, the loss function of the molecular prediction model further comprises: a difference between a predetermined gradient and a gradient of a predicted value of the sample molecular energy obtained based on the sample molecular structure relative to the sample molecular structure.
